(19) United States
(12) Patent Application Publication
(10) Pub. No.: US 2010/0258446 A1
(43) Pub. Date: Oct. 14, 2010

(54) SYSTEMS INCLUDING NANOTUBULAR ARRAYS FOR CONVERTING CARBON DIOXIDE TO AN ORGANIC COMPOUND

(75) Inventors: Susanta Mohapatra, Lexington, KY (US); Manoranjan Misra, Reno, NV (US)

Correspondence Address: WOOD, HERRON & EVANS, LLP, 2700 CAREW TOWER, 441 VINE STREET, CINCINNATI, OH, 45202, US

(73) Assignee: Board of Regents of the Nevada System of Higher Education, on behalf of the University of Nevada, Reno, NV (US)

(21) Appl. No.: 12/754,269

(22) Filed: Apr. 5, 2010

Related U.S. Application Data
(60) Provisional application No. 61/166,354, filed on Apr. 3, 2009.

(51) Int. Cl. C25B 9/04; B01J 19/12; C07C 29/00

(52) U.S. Cl. 205/340; 422/186.04; 204/230.2; 204/157.6; 204/157.9; 977/700; 977/773

(57) ABSTRACT

A system including nanostructure arrays for converting carbon dioxide to an organic compound, e.g., methanol, which does so, for example, without any external electric energy. In one embodiment, the system for converting carbon dioxide to an organic compound includes an array of nanotubes, which include nanoparticles of an electron mediator, e.g. palladium, dispersed on a surface of the nanotubes, and an electrically conductive fluid. The array of nanotubes is at least partially immersed in the electrically conductive fluid. The system further includes a light source that irradiates the array of nanotubes, a source of carbon dioxide, and an inlet for delivering the carbon dioxide to the electrically conductive fluid whereat at least a portion of the carbon dioxide is converted to a different organic compound, such as methanol, via contact with an irradiated array of nanotubes. In one example, the array is an ordered array of titania nanotubes.

### CROSS REFERENCE TO RELATED APPLICATIONS

This application claims the benefit of U.S. Provisional Application No. 61/176,355, filed Apr. 3, 2009, the disclosure of which is hereby incorporated by reference herein in its entirety.

### TECHNICAL FIELD

The present invention relates generally to nanostructure arrays and uses thereof for converting carbon dioxide to an organic compound.

### BACKGROUND

Global warming due to emission of carbon dioxide (CO₂) is a serious environmental concern. The atmospheric concentration of CO₂ is about 384 ppm by volume and about 3×10¹² tonnes by weight. Burning of fossil fuels, such as coal, gas, and oil, and deforestation are the leading causes for the increase in the anthropogenic CO₂. Based on 2006 data, more than 32×10⁹ tonnes of CO₂ are released per year worldwide from fossil fuels.

Pumping CO₂ from fossil fuel power plants into deep ocean basins is one of the most discussed disposal options. Another potential option is converting this greenhouse gas into alternate fuels. For example, CO₂ feedstock can be converted into fuels of solid, liquid, or gas phase by chemical, electrolytic, photocatalytic, or photo-electrocatalytic methods. However, reduction of CO₂ to fuel form of any phase requires energy, which can make the process unattractive both from an economical standpoint and because the process can create yet more CO₂. Similarly, processes that are carried on at high temperatures or pressures can be unattractive.

Electrolytic conversion of CO₂ into various forms such as methane, ethane, ethylene, methanol, ethanol, n-propanol, formic acid, and formaldehyde has been widely reported. The electrolytic reduction of CO₂ to methanol requires six electrons according to the reaction set forth in Eq. 1:

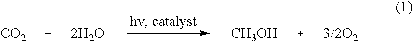

Electrolytic conversion of CO₂ to useful products typically suffers from a number of limitations. For example, the reaction of Eq. 1 can be kinetically limited because of the low solubility of CO₂ in water. In addition, in aqueous solutions, hydrogen evolution can compete with methanol formation. Also, the reaction of Eq. 1 is energetically demanding and involves uncontrolled intermediates.

It also appears that processes to date for converting CO₂ to methanol rely on indirect power sources to supply the necessary energy, rather than using more direct energy sources, such as solar energy. Although there appears to have been some investigation of solar conversion of CO₂ to methanol using UV-irradiated titanium dioxide (also referred to as titania) nanoparticles, the process, for example, appears to have very low CO₂ to methanol conversion (0.5 mmol/g-cat-hr) when using titania (TiO₂) nanoparticles under UV irradiation.

It would thus be beneficial to provide a system including nanostructure arrays for converting carbon dioxide to an organic compound, which overcomes the aforementioned drawbacks and does so, for example, without any external electric energy.

### SUMMARY

In one embodiment, a system is disclosed for converting carbon dioxide to an organic compound. The system includes an array of nanotubes, which include nanoparticles of an electron mediator, e.g. palladium, dispersed on a surface of the nanotubes, and an electrically conductive fluid. The array of nanotubes is at least partially immersed in the electrically conductive fluid. The system further includes a light source that irradiates the array of nanotubes, a source of carbon dioxide, and an inlet for delivering the carbon dioxide to the electrically conductive fluid whereat at least a portion of the carbon dioxide is converted to a different organic compound, such as methanol, via contact with an irradiated array of nanotubes. In one example, the array is an ordered array of titania nanotubes.

In another embodiment, the system includes an anode, a cathode, and an electrically conductive material. The anode includes an array of nanotubes, e.g., titania nanotubes. The cathode includes an electrically conductive material and also cooperates with the anode to receive electrons therefrom. The anode and cathode are at least partially immersed in the electrically conductive fluid. The system further includes a light source that irradiates at least the anode, a source of carbon dioxide, and an inlet for delivering the carbon dioxide to the electrically conductive fluid whereat at least a portion of the carbon dioxide is converted to a different organic compound, such as methanol, via contact with the cathode, anode, or both. In one example, the cathode is a gas diffusing cathode. In another example, the system carries out conversion of CO₂ to methanol, without supply of any external electric energy.

In another embodiment, a method for converting carbon dioxide to an organic compound is disclosed. The method includes irradiating an array of nanotubes at least partially immersed in an electrically conductive fluid. The array of nanotubes includes nanoparticles of an electron mediator, e.g. palladium, that are dispersed on a surface of the nanotubes. The method further includes delivering carbon dioxide to the electrically conductive fluid whereat at least a portion of the carbon dioxide is converted to a different organic compound, such as methanol, via contact with the irradiated array of nanotubes. In one example, the array is an ordered array of titania nanotubes.

In another embodiment, the method includes irradiating an anode including an array of nanotubes, e.g., titania nanotubes. The method also includes supplying electrons from the irradiated anode to a cathode including an electrically conductive material. The anode and the cathode are at least partially immersed in the electrically conductive fluid. The method further includes delivering carbon dioxide to the electrically conductive fluid whereat at least a portion of the carbon dioxide is converted to a different organic compound, such as methanol, via contact with the cathode, anode, or both.

There are additional features and advantages of the subject matter described herein. They will become apparent as this specification proceeds.

In this regard, it is to be understood that this is a brief summary of varying aspects of the subject matter described herein. The various features described in this section and below for various embodiments may be used in combination or separately. Any particular embodiment need not provide all features noted above, nor solve all problems or address all issues in the prior art noted above. Additional features of the present disclosure are described in the appended claims.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a schematic diagram of a system for photocatalytically converting carbon dioxide (CO₂) to methanol (CH₂OH) using an array of titania (TiO₂) nanotubes with nanoparticles of palladium (Pd) dispersed on a surface thereof in accordance with an embodiment of the invention;

FIG. 2 is a block flow diagram of a method of converting CO₂ to methanol to dimethyl ether in accordance with an embodiment of the invention;

FIG. 3 is a schematic diagram illustrating a proposed mechanism by which a cross-sectioned Pd-sensitized titania nanotube convert CO₂ to methanol in the presence of light;

FIG. 4 is a schematic diagram of a system for photoelectrochemically converting carbon dioxide to methanol using a cathode and an anode including an array of titania nanotubes with nanoparticles of palladium dispersed on a surface thereof in accordance with an embodiment of the invention;

FIG. 5 is a schematic diagram of a system for photoelectrochemically converting carbon dioxide to methanol to dimethyl ether in accordance with an embodiment of the invention; and

FIG. 6 is a schematic diagram illustrating the Pd-sensitized titania nanotubes in accordance with an embodiment of the invention.

### DETAILED DESCRIPTION OF SPECIFIC EMBODIMENTS

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, as well as A and B together. Although methods and materials similar or equivalent to those described herein can be used in the practice or testing of the present disclosure, suitable methods and materials are described herein. The disclosed materials, methods, and examples are illustrative only and not intended to be limiting.

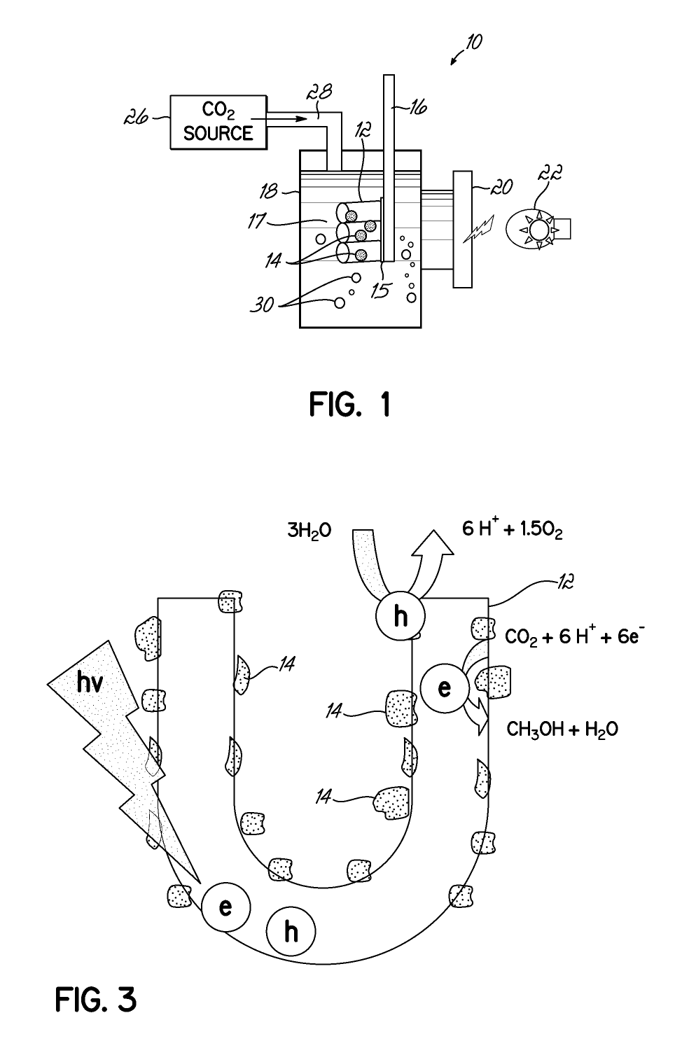

FIGS. 1-6 depict various embodiments of systems and methods directed to photocatalytic or photoelectrochemical conversion of carbon dioxide to an organic compound, such as methanol, using nanostructured arrays. While the systems discussed herein describe the conversion of carbon dioxide to methanol, which may itself be further converted to dimethyl ether, it should be understood that other organic liquid compounds may be derived from carbon dioxide, which themselves may further be converted to various organic liquid compounds.

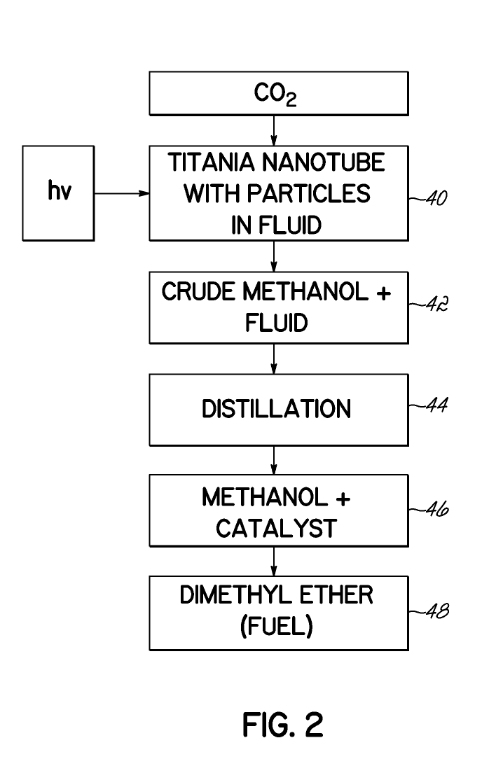

With specific reference now to FIGS. 1 and 2 and in accordance with embodiments of the invention, a system 10 and method is disclosed for photocatalytically converting carbon dioxide to methanol, which further optionally is converted to dimethyl ether. The system 10, as shown in FIG. 1, includes an array of titania nanotubes 12 having nanoparticles of an electron mediator 14, e.g., palladium, dispersed on a surface of the nanotubes 12. The array of nanotubes 12 may be formed on a titanium substrate 15, for example, which can be secured to a rod 16, and immersed, either wholly or partially, in an electrically conductive fluid 17, such as a dilute sulfuric acid solution (e.g., pH 1.5), all of which is contained in a cell 18 having a quartz window 20 for light illumination. The system 10 further includes a light source 22, such as a solar light, e.g., the sun, that irradiates the array of nanotubes 12 with light, e.g., visible light. The system 10 also includes a source of carbon dioxide 26 and an inlet tube 28 for delivering the carbon dioxide 30 to the electrically conductive fluid 17 whereat at least a portion of the carbon dioxide 30 is converted to methanol via contact with an irradiated array of palladium-sensitized titania nanotubes 12. In one example, the source of the carbon dioxide 30 is a fossil fuel, e.g., coal, which upon burning or combustion at a plant, for example, releases carbon dioxide as a byproduct. A gas outlet (not shown) for the generation of gases within the cell may also be provided, as well as any additional inlets or outlets, such as for movement of the electrically conductive fluid 17 into or out of the system 10, as is desired or necessary.

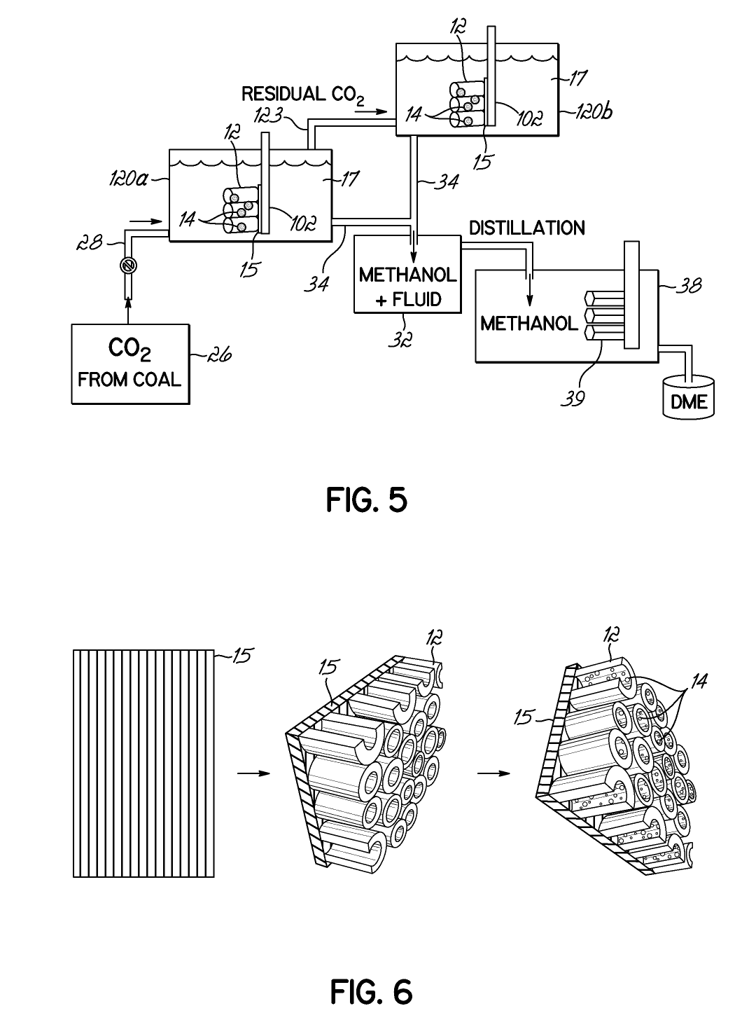

The system 10 also can be further provided with a distillation unit 32 (See FIG. 5) to allow for separation of the methanol from the electrically conductive fluid 17. For example, the cell 18 can be connected to the distillation unit 32, such as by tubing 34 or other suitable connections, so as to transfer the methanol/electrically conductive fluid mixture therefrom. After distillation, the distilled methanol can be transferred to another cell 38 including a catalyst 39, such as an aluminum oxide catalyst, whereat at least a portion of the methanol can be converted via the catalyst 39 to another organic compound, e.g., dimethyl ether, for use as a fuel source, all of which is more fully discussed below.

With specific reference now to FIG. 2, the method for photocatalytically converting carbon dioxide 30 to an organic compound is diagrammatically illustrated. In this method, the system 10 of FIG. 1 converts carbon dioxide 30 to methanol, which is further optionally converted to dimethyl ether. The method includes irradiating with light (hv) the immersed array of titania nanotubes 12 using light source 22, such as a solar light simulator or the sun, as depicted by block 40. In one example, the titania nanotubes 12 are continuously irradiated. Carbon dioxide 30 is also delivered to the dilute sulfuric acid solution 17 such as to saturate the sulfuric acid solution 17, as also depicted by block 40, whereat at least a portion of the carbon dioxide 30 is converted to methanol via contact with the irradiated array of nanotubes 12. In one example, the fluid 17 may be continuously purged with carbon dioxide 32 at a rate of ˜10 cc/min The carbon dioxide 30, as shown in FIG. 6, may be transported from a coal plant as a byproduct of coal combustion to the sulfuric acid solution 17 of the system 10 via inlet tube 28.

Concerning the conversion of carbon dioxide 30 to methanol and without intending to be bound by theory, it is understood that the anodic and cathodic reactions occur at different sites of the palladium-sensitized TiO₂ nanotubes 12 to convert the carbon dioxide 30 to methanol, as is illustrated in FIG. 3. In particular, when the array of TiO₂ nanotubes 12 is illuminated with solar light (hv), electron hole pairs (h) are generated. These holes (h) are consumed by the water oxidation reaction: 3H₂O+6H+→6H⁺+1.5O₂. During this process, H⁺ ions are generated. The electrons (e) are trapped by the palladium nanoparticles 14 and participate in the CO₂ reduction process: CO₂+6H⁺→CH₃OH+H₂O. Oxygen evolution preferentially occurs at the TiO₂ anodic reaction and the corresponding cathodic reaction of methanol formation is considered to occur at the palladium sites. And since no external energy is required, the energy savings are on the order of 0.1-0.24 kWh/mole of CO₂.

With continuing reference now to FIG. 2, the carbon dioxide 30 converts to methanol, as depicted by box 42, which begins to collect in the electrically conductive fluid 17. Thereafter, the methanol can be removed from the electrically conductive fluid 17 through distillation, as depicted by box 44. In one example, methanol produced by the system 10 can be collected by vacuum distillation (˜40° C.), as is known in the art. The methanol and electrically conductive fluid mixture may be transferred to and collected in distillation unit 32. The methanol is subsequently distilled from the solution. If the methanol is desired to be converted to another organic substance, such as dimethyl ether, it can be subjected to additional processing steps. For example, as further depicted in block 46 of FIG. 2, the methanol can combined with catalyst 39, such as a nanotubular solid-acid catalyst (α-Al₂O₃ or AlMg mixed metal oxides), so as to produce dimethyl ether, as depicted in block 48. In one example, the catalyst is anodized aluminum oxide nanotubes. The dimethyl ether can be used as a valuable fuel, for example, by replacing liquid petroleum gas (LPG) or by mixing with gasoline. Aside from dimethyl ether, it should be understood that other organic compounds may be produced as desired, such as acetic acid, formaldehyde, and the like, using suitable catalysts and methods known in the art.

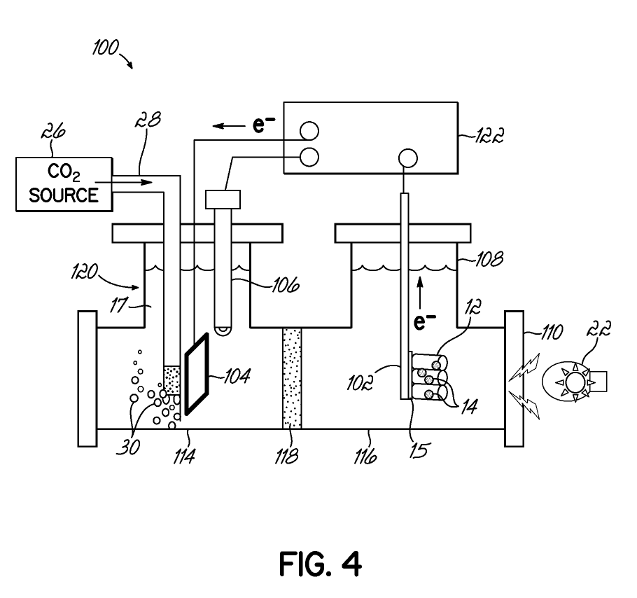

In accordance with other embodiments of the invention and with reference now to FIGS. 4 and 5, a system 100 and method are disclosed for photoelectrochemically converting carbon dioxide 30 to methanol, which further is optionally converted to dimethyl ether. The system 100 includes an anode 102, a cathode 104, a reference electrode 106, and electrically conductive fluid 30, such as a dilute sulfuric acid solution (e.g., pH 4.5), all of which is contained in a cell 108 having a quartz window 110 for light illumination. The anode 102, cathode 104, and reference electrode 106 are immersed, either wholly or partially, in the electrically conductive fluid 17. The cathode 104 is shown situated in a separate compartment 114 that is connected to anode compartment 117 through a porous glass frit 118. The anode 102, cathode 104, and reference electrode 106 together, in part, define a three-electrode cell 120.

The anode 102 includes an array of nanotubes, such as the array of titania nanotubes 12 on substrate 15 optionally including nanoparticles of electron mediator 14, e.g., palladium, dispersed on a surface of the nanotubes 12. The cathode 104 includes an electrically conductive material, e.g., titanium, and cooperates with the anode 102 via a potentiostat 122 to receive electrons (e) therefrom. The system 100 further includes light source 22, such as a solar light, e.g., the sun, that irradiates the array of nanotubes 12 with light, e.g., visible light. The system 100 also includes the source of carbon dioxide 26 and inlet tube 28 for delivering the carbon dioxide 30 to the electrically conductive fluid 17 whereat at least a portion of the carbon dioxide 30 is converted to methanol via contact with the cathode 104, anode 102, or both 102, 104. In one example, the system 100 carries out conversion of carbon dioxide 30 to methanol, without supply of any external electric energy. A gas outlet (not shown) for the generation of gases within the cell may also be provided, as well as any additional inlets or outlets, such as for movement of the electrically conductive fluid 17 into or out of the system 100, as is desired or necessary.

As shown in FIG. 5, the system 100 may be further provided with distillation unit 32 so as to separate the methanol from the electrically conductive fluid 17. After distillation, the distilled methanol can be transferred to cell 38 including catalyst 39, such as an aluminum oxide catalyst, whereat at least a portion of the methanol can be converted via the catalyst 39 to another organic compound, e.g., dimethyl ether, for use as a fuel source, as more fully discussed below. Also, in this embodiment, two three-cell electrodes 120a and 120b are arranged in parallel fashion to increase the carbon dioxide conversion to methanol. The second three-cell electrode 120b is connected to the first three cell electrode 120a via residual CO₂ tube 123. Each of the three-cell electrodes 120a, 120b is connected to the distillation unit 32, such as by tubing 34 or other suitable connections, so as to transfer the methanol/electrically conductive fluid mixture. And while two three-cell electrodes 120a, 120b are shown, it should be understood that one or more than two three-cell electrodes may be utilized. In addition, it should be understood that the system 10 of FIG. 1 may suitably replace system 100 therein for conversion of carbon dioxide 30 to methanol and further conversion thereof to dimethyl ether.

With continuing reference to FIGS. 4 and 5, the method for photoelectrochemically converting carbon dioxide 30 to an organic compound is schematically illustrated. In this method, the system 100 converts carbon dioxide 30 to an organic compound, i.e., methanol, which is further optionally converted to another organic compound, i.e., dimethyl ether. The method includes irradiating the immersed anode 102, which includes the array of titania nanotubes 12 optionally including nanoparticles of palladium 14, with solar light source 22, such as a solar light simulator or the sun. In one example, the titania nanotubes 12 are continuously irradiated. The cathode 104 cooperates with the anode 102 via potentiostat 122 to receive electrons (e) therefrom as a result of photocatalytic reactions at the anode 102. Carbon dioxide 30 also is delivered to the dilute sulfuric acid solution 17 of the first cell 120a, with residual carbon dioxide 30 being delivered to the dilute sulfuric acid solution 17 of the second cell 120b, so as to saturate the sulfuric acid solution 17 whereat at least a portion of the carbon dioxide 30 is converted to methanol via contact with the cathode 104. In one example, the fluid 17 may be continuously purged with carbon dioxide 30 at a rate of ˜10 cc/min. The carbon dioxide 30 may be transported from a coal plant as a byproduct of coal combustion to the sulfuric acid solution 17 of the system 100 via inlet tube 28. The carbon dioxide converts to methanol, which begins to collect in the electrically conductive fluid 17 of the first and second cells 120a, 120b. The methanol may be removed from the electrically conductive fluid 17 through distillation.

Without intending to be bound by theory, inherent defects in the cathode materials may act as preferential sites for CO₂ adsorption, which can enhance reaction kinetics. It is also contemplated that at least a portion of the carbon dioxide 30 can be converted to methanol via contact with the anode 102 of system 100.

With continuing reference to FIG. 5, the methanol and electrically conductive fluid mixture is collected in distillation unit 32. The methanol is subsequently distilled from the solution. In one example, methanol produced by the system 100 can be collected by vacuum distillation (˜40° C.), as is known in the art. If the methanol is desired to be converted to another organic substance, such as dimethyl ether, it can be subjected to additional processing steps. In particular, the methanol may be combined with a suitable dehydration catalyst 39, such as such as nanotubular solid-acid catalyst (α-Al₂O₃ or AlMg mixed metal oxides), to produce dimethyl ether. In one example, the catalyst 39 is anodized aluminum oxide nanotubes. The dimethyl ether can be used as a valuable fuel, for example, by replacing liquid petroleum gas (LPG) or by mixing with gasoline. Aside from dimethyl ether, it should be understood that other organic compounds may be produced as desired, such as acetic acid, formaldehyde, and the like, using suitable catalysts and methods known in the art.

While the array of nanotubes 12 is described above in both systems as an array of titania (TiO₂) nanotubes, it should be understood that the nanotubes 12 may be made from a variety of other materials. Other suitable non-limiting materials can include silicon, zirconium, aluminum, cerium, yttrium, neodymium, iron, antimony, silver, lithium, strontium, barium, ruthenium, tungsten, nickel, tin, zinc, tantalum, molybdenum, chromium, and mixtures thereof. Suitable compounds include transition metal chalcogenides or oxides, including mixed metal and/or mixed chalcogenide and/or mixed oxide compounds. In particular examples, the nanotubular structure can be made from one or more of zinc oxide, gallium nitride, indium oxide, tin dioxide, magnesium oxide, tungsten trioxide, and nickel oxide. In another example, one or more materials from which the nanotubular structure is made are semiconductors. The nanotubes 12, such as the titania nanotubes, also may be doped or modified, such as with carbon or nitrogen.

With specific reference to FIG. 6, the nanotubes 12 generally are solid structures having a hollow core and a cross sectional diameter of between about 0.5 nm to about 500 nm. In another example, the cross sectional diameter of the nanotube 12 is between about 0.5 nm and about 200 nm. While a nanotubular type structure is disclosed herein, it is understood that other nanostructures may be utilized, such as wires and nanorods, which may have cross sectional diameters of between about 0.5 nm to about 500 nm. In certain embodiments, the cross sectional dimension of the nanostructure is relatively constant. In other embodiments, the cross sectional dimension of the nanostructure can vary, e.g., the nanotubes have a taper. The length or size of the nanotubes 12 can range from about 10 nm to 1000 microns. In another example, the size of the nanotubes 12 can range from about 500 nm to 100 microns.

The titania nanotubes 12 may be formed on the titanium substrate 15 (e.g., a titanium foil), then loaded with the nanoparticles of the catalytic electron mediator 14, as is discussed in more detail further below. In general, nanostructures, such as nanotubes, may be formed on or attached to substrates made of generally inert materials and which typically are insulating. The substrate 15 is typically selected to be stable during the process(es) by which the nanostructures are formed or placed on the substrate 15. For example, in some methods, the substrate 15 is capable of withstanding relatively high temperatures, such as at least about 500° C. Other non-limiting examples of substrate materials include ceramics, glasses, such as silica or soda-lime glass, quartz, alumina, silica, and insulating polymers.

The band gap of the nanostructures, e.g., the titania nanotubes 12, may be engineered, according to methods known in the art, to be at least about 2 eV, such as between about 2 eV and about 5 eV, between about 2 eV and about 4 eV, or between about 2 eV and about 3 eV. In one example, the band gap is between about 2.0 ev and about 2.2 ev. In yet another example, the titania nanotube 12 has a band gap of less than about 4 eV. Titania nanotubes 12 with this bandgap can harvest solar light in the visible region. In addition, the titania nanotubes 12 can have a resistivity lower than about 10⁻³ Ωm, such as less than about 10⁻⁶ Ωm or less than about 10⁻⁷ Ωm, such as between about 10⁻¹⁴ Ωm and about 10⁻¹⁰ Ωm or between about 10⁻¹² Ωm and about 10⁻⁶ Ωm. In one example, the nanostructures have a resistivity of about 10⁻¹² Ωm.

The electron mediator 14 may be a metal or a semiconductor material. Aside from palladium, other suitable non-limiting examples include platinum, niobium, molybdenum, tantalum, tungsten, rhenium, ruthenium, irridium, a metal carbide, or an oxynitride. The size of the nanoparticles of the electron mediator 14 can range from 1 nm to 100 nm. In one example, the nanoparticles can range from 5 nm to 100 nm.

The electrically conductive fluid 17 may be an ionic solution (aqueous), which may be formed via a salt, an acid, or a base, or an organic solvent. In another example, the electrically conductive fluid 17 includes an aprotic solvent, such as acetonitrile, glycols, or imidazolium salts. Reducing the amount of water present may reduce hydrogen evolution and improve electrode stability. Aside from a dilute sulfuric acid solution, other suitable non-limiting examples of the electrically conductive fluid 17 include an imidazolium salt solution or an organic carbonate.

As discussed above, the light source 22 may be a solar light source, e.g., the sun, which irradiates the array of nanotubes with visible light. In one example, the light source 22 can be a solar simulator that simulates the sun's spectra. In general, the light source 22 may be any light source that provides the appropriate type of light suitable for converting carbon dioxide to the desired organic compound. In another example, the light source 22 may be an ultraviolet light to irradiate the nanotubes with ultraviolet light. In still another example, the light source 22 provides only a specific wavelength, such as from the visible and/or uv spectra.

The source of carbon dioxide 26, as earlier mentioned, may be a fossil fuel, e.g., coal, which upon burning or combustion releases carbon dioxide as a byproduct. Other combustible fossil fuels that can produce carbon dioxide include oil and gas. In addition, a number of other specialized industrial production processes and product uses, such as mineral production, metal production, and the use of petroleum-based products, can also lead to CO₂ emissions and be a source of carbon dioxide 26, which can be directed to the electrically conductive fluid via an inlet tube, for example.

The cathode 104 of system 100 includes an electrically conducting material, such as a semiconductor material or a metal, such as titanium. Examples of suitable materials include aluminum, gold, indium-tin-oxide, fluorine-doped tin oxide, chromium, nickel, tungsten, palladium, platinum, ruthenium, or other metals, metal alloys, or mixtures thereof. In one example, the cathode 104 is a titanium cathode. In another example, the cathode 104 defines a gas-diffusing p-type semiconductor including a titanium dioxide substrate. Other suitable cathodes include p-type semiconductor nanostructures formed, in some examples, using a pulse-reverse electrodeposition route in low-temperature aprotic or ionic electrolytes. In another example, suitable semiconductor materials include ZnTe, Cd_{0.92}Zn_{0.08}Te, AlSb, GaP, or InP that can be formed on TiO₂ nanotubular templates.

The reference electrode 106 is a Ag/AgCl electrode but may be any suitable reference electrode, as is known in the art.

The anode 102, cathode 104, and reference electrode 106 contacts can be connected to electrical devices, such as resistivity measurement apparatus, such as by the potentiostat 122. Connections can be formed through any suitable means, such as silver paste/epoxy or wire bonding, such as ball bonding or wedge bonding. In one embodiment, an external source of energy (not show such as electrical energy, may be supplied to system.

Nanostructure Synthesis

Presently disclosed embodiments concern using nanostructures, particularly arrays of nanotubes 12, which may include nanoparticles of an electron mediator 14, to convert carbon dioxide 30 to organic products, such as methanol or dimethyl ether. These nanostructures, e.g., the nanotubes, may be formed by any suitable method. The nanostructures may be relatively homogenously disposed on a substrate. In other examples, the nanostructures may be unevenly distributed on a substrate or distributed in discrete zones. For example, the array may be formed in a stepped structure, such as to aid in uniformity of the nanostructures by forming the nanostructures in discrete portions, whose properties thus may be more accurately controlled.

In one example, formation of nanotubes involves anodizing a metal or metal alloy source, such as a titanium foil, in a suitable electrolytic solution. Suitable titanium foils can be obtained from commercial sources or can be prepared by various methods, such as sputtering. In some examples, the metal source has a thickness suitable to produce a desired amount of oxide, or other substance, while retaining a sufficient amount of metal to aid in handling, durability, or conduction which, in some embodiments, is at least about 300 nm, such as between about 300 nm and about 10 mm or between about 1000 nm and about 4 mm. In a specific example, the metal source has a thickness of about 2 mm.

Prior to anodization, the metal source can be cleaned, such as by washing the source in an organic solvent, such as acetone, methanol, isopropanol, or mixtures thereof (including aqueous mixtures), optionally with sonication. The metal source can be further rinsed with water, such as deionized water, and dried.

Prior to use, the substrate may be subjected to one or more pretreatment steps, such as cleaning steps. Cleaning steps can include treating the substrate with a solvent, such as an organic solvent, to remove impurities present on the surface of the substrate. In a particular example, the solvent is acetone. Ultrasonication may also be used to clean the surface of the substrate.

The dimensions of the substrate can be tailored to a particular application, such as the nanostructure composition, size, desired detection limit, and other components of an apparatus with which the nanostructure array will be used. In particular examples, the substrate has a thickness of between about 0.25 mm and about 2 mm, such as between about 0.5 mm and about 1 mm. Additional materials can be placed on the substrate, such as to facilitate handling of the structure or to aid in subsequent processing steps. For example, in some methods, a layer of aluminum is deposited on the substrate prior to deposition of the material from which the nanostructures will be formed.

In some embodiments, the electrolytic solution includes at least one acid, such as a solution of acetic acid, chromic acid, phosphoric acid, oxalic acid, hydrofluoric acid, or mixtures thereof. In more specific examples, the acidic solution also includes a fluoride compound, such as hydrogen fluoride or alkali fluorides, such as sodium fluoride or potassium fluoride. The solution includes at least about 0.1 wt % of fluoride compounds in some examples, such as about 0.5 wt % of one or more fluoride compounds. In other examples, a basic electrolytic solution is used, such as a solution of potassium hydroxide. The electrolyte solution can include other substances.

In various examples, the anodization potential is between about 1 V and about 50 V, such as between about 5 V and about 20 V or about 10 V. Constant anodization voltage can be used to produce nanotubes having a relatively constant diameter. Ramped or stepped voltages can be used to produce shaped nanotubes, such as tapered conical nanotubes. Pulsed electrolysis can also be used.

The temperature of the anodization process can also affect the properties of the nano structures, such as the wall thickness of nanotubes. Lower temperatures typically produce nanotubes having thicker walls. Typical temperatures are between about 5° C. and about 75° C., such as between about 15° C. and about 50° C. The pH of the electrolyte solution is typically between about 0.1 to about 7, such as between about 3 and about 5.

In at least some examples, the bath is agitated during all or a portion of the anodization process. Suitable means of agitation include magnetic or mechanical stirring. Ultrasonication can also be used to agitate the electrolyte solution.

Anodization is carried out for a sufficient time to form nanostructures having a desired length or other property, such as between about 1 minute and about 24 hours. Amorphous structures produced by such methods can be crystallized by annealing the nanostructures, such as by heating the nanotubes at a suitable temperature and a period of time of about 200° C. to about 1200° C. for about 10 minutes to about 7 hours.

According to another disclosed embodiment, nanotubes are prepared by treating a suitable metal oxide with alkali. For examples, titanium nanotubes may be prepared by treating titanium dioxide with about 13 wt % to about 65 wt % alkali, such as alkali or alkaline earth metal hydroxides, including sodium hydroxide and potassium hydroxide, at a temperature of between about 18° C. and about 170° C. In such examples, the diameter of the nanotubes is typically between about 5 mu and about 80 nm. The thickness of the nanotubes walls is typically between about 2 nm and about 10 nm, while the length of the nanotubes is typically between about 50 nm and about 150 nm. The titanium dioxide particles, in specific examples, have an average particle diameter of between about 2 nm and about 100 nm, such as between about 2 mu and about 30 nm. The properties of the resulting nanotubes, such as crystallinity and catalytic or electrical properties, can be modified by heating the nanotubes at about 200° C. to about 1200° C. for about 10 minutes to about 7 hours.

Another disclosed embodiment for forming nanotubes involves alkaline treatment of titanium oxide, titanium oxyhydroxide, or titanium hydroxide, followed by ionic exchange, such as by acid treatment, to obtain materials that include hydrogen titanates. The alkaline solution typically has a concentration of about 1 M to about 50 M, such as about 5 M to about 50M. Examples of alkaline materials which may be used to generate the solution include ammonium hydroxide, potassium hydroxide, sodium carbonate, and sodium hydroxide, as well as other alkali or alkaline earth metal hydroxides.

The alkaline titanium solution is heated, optionally with stirring, at about 50° C. to about 180° C., at a pressure of about 1 atm to about 150 atm, for about 1 hour to about 100 hours. The resulting product is treated with a dilute acid solution, such as about a 0.1 M to about 1M solution of an ammonium salt, such as ammonium carbonate or ammonium chloride, boric acid, chlorhidric acid, fluoric acid, nitric acid, phosphoric acid, or sulfuric acid until the pH of the solution is between about 1 and about 7, such as between about 2 and about 4. The solution is then held for about 1 hour to about 24 hours at a temperature of about 20° C. The resulting solid is then separated from this mixture, washed, and dried at temperature of about 60° C. to about 120° C. The structure of the material may be further adjusted by heating the material at a temperature between about 200° C. and about 500° C. in an atmosphere of one or more of oxygen, nitrogen, argon, hydrogen, and helium at a flow rate of about 0.1 l/min to about 1 l/min.

Another disclosed embodiment for producing nanostructures involves forming a sol solution of a metal oxide or similar material, such as titanium oxide, in water, optionally with a co-solvent, such as a lower (e.g. 10 carbon atoms or fewer) alcohol, such as methanol or ethanol. The oxide is typically included in a concentration of about 2 wt % to about 50 wt %. The oxide particles typically have an average particle diameter of between about 2 nm and about 100 nm.

The resulting mixture is treated with a source of peroxide, such as hydrogen peroxide, to produce a peroxometal compound, such as peroxotitanic acid. In order to encourage all of the metal oxide to react with the peroxide, the peroxide typically is added in at least a 1:1 weight ratio to the metal oxide and is let stand for about 30 minutes to about 24 hours to dissolve the metal oxide. The mixture is then heated at about 50° C. to about 300° C., optionally with ammonium hydroxide, an organic base, or mixtures thereof.

An alkali metal hydroxide, such as LiOH, NaOH, KOH, RbOH, CsOH, or a mixture thereof, is added to the resulting solution in a ratio of alkali hydroxide to metal oxide of about 1:1 to about 30:1 or about 2:1 to about 25:1. This mixture is then heated at about 50° C. to about 350° C. The resulting material can be washed. In some examples, the wash is an acidic solution, such as a solution of a mineral acid, for example, hydrochloric acid or nitric acid. This material is then treated with a cation source at a ratio of cation source to metal oxide of about 1:1 to about 30:1 or about 2:1 to about 25:1 at a temperature of about 50° C. to about 350° C., such as about 80° C. to about 250° C. The cation source may be, for example, acids, non-alkali salts, and organic bases. Suitable acids include mineral acids, such as hydrochloric acid, nitric acid, and sulfuric acid. Organic acids may also be used, such as acetic acid, citric acid, glycolic acid, glycidic acid, maleic acid, malonic acid, and oxalic acid. Examples of organic bases usable in this technique include ammonium hydroxide; amines, such as monoethanolamine, diethanolamine, and triethanolamine; quaternary ammonium salts, such as tetramethylammonium salts; and derivatives or mixtures thereof. Examples of ammonium salts include ammonium acetate, ammonium chloride, ammonium nitrate, and ammonium sulfate. The cation-treated material is heated at about 50° C. to about 350° C., such as about 80° C. to about 250° C. The resulting material is then reduced in an inert gas, such as nitrogen, helium, neon, argon, krypton, xenon, or radon; along with a reducing gas, such as an amine, ammonia, hydrazine, or pyridine; or a hydrocarbon, such as methane, ethane, or propane, or mixtures thereof. The reduction is typically carried out at a temperature of about 100° C. to about 700° C.

Suitable nanostructures can also be produced by electrospinning, during which a core solution of an extractable or otherwise removable material, such as mineral oil, and a sheath solution of sheath material are subject to a high voltage. The solutions are thus charged and forced through a spinneret. In some embodiments, the sheath solution includes a viscosity modifying agent, such as a polymer. Suitable polymer materials include poly(vinyl pyrrolidone), polystyrene, polypropylene, polyethylene, polyvinyl chloride, polyvinylidene chloride, polyvinyl acetate, nylon polymers, polyurethane, and mono or poly functional (meth)acrylates. Additional polymer materials are disclosed in paragraph 51 of U.S. Patent Publication 2006/0223696, which is incorporated by reference herein.

Materials which can be used to form suitable sheaths include sol-gel precursors such as metal alkoxides; metal halides, such as metal chlorides, TiCl₄ for example; metal hydroxides; metal sulfates, such as Ti(SO₄)₂; metal acetylacetonates; and derivatives or combinations thereof. The metal alkoxides and metal acetylacetonates are typically tetrafunctional, the organic component being, in some examples, linear or branched C₁-C₁₂ alkoxides, such as methoxide, ethoxide, isopropoxide, n-butoxide, 2-butoxide, t-butoxide, n-hexoxide, 2-ethylhexoxide, 2-methoxy-1-ethoxide, acetylacetone, and mixtures thereof. In a particular example, the sheath material is Ti(OiPr)₄ (titanium tetraisopropoxide). Suitable solvents for these sols include lower alkyl alcohols (e.g. 10 carbon atoms or fewer), such as methanol, ethanol, isopropanol, n-butanol, cyclohexanol; solvents containing a carbonyl group, including aldehydes, such as acetone and cyclohexanone, ketones, such as methyl ethyl ketone, and esters, such as ethyl acetate; aromatic solvents, such as benzene, phenol, benzyl alcohol, and toluene; halogenated solvents, such as chloroform, methylene chloride, carbon tetrachloride, trichloroethane, hexafluoroisopropanol, and hexafluoroacetone; ethers, such as diethylether; other solvents, such as N-methylpyrrolidone, 1,3-dioxolan, acetonitrile, N-methyl-morpholine-N-oxide, tetrahydrofuran, N,N-dimethylformamide, pyridine, 1,4-dioxane, acetic acid, formic acid; and mixtures thereof.

In at least some embodiments, the core and sheath solutions are selected to be at least substantially immiscible. In yet further embodiments, the core and sheath solutions are at least somewhat miscible. The miscibility of the solvents can be used to adjust the porosity of the resultant nanostructures. For example, a core solution of polystyrene in N,N-dimethyl formamide and tetrahydrofuran can be used with an ethanolic solution of the structural material to produce highly porous nanostructures.

The core solution can be modified to include agents to coat the lumen of the sheath. For example, the core solution can include any of the sensitizing agents discussed in this disclosure. The properties of the nanotube surfaces can also be modified by adding appropriate agents to the core and sheath solutions. For example, alkylsilanes can be used to make a surface more hydrophobic.

Spinnerets suitable for use in forming nanostructures have a number of configurations, such as syringe-in-syringe arrangements; concentric spinnerets, such as using nested capillaries; or micro channels, such as branched micro channels. Suitable devices are disclosed in Srivasta, et al., “Electrospinning of hollow and core/sheath nanofibers using a microfluidic manifold,” Microfluid nanofluid DOI 10.1007/s10404-007-0177-0; Li et al., “Direct Fabrication of Composite and Ceramic Hollow Nanofibers by Electrospinning,” Nano Letters 5(4) 933-938 (2004); and U.S. Patent Publication 2006/0226580; each of which is incorporated by reference herein in its entirety.

The spinning conditions, including size and configuration of the spinnerets, the composition of the feed solutions, applied voltage, and solution feed rates can be adjusted to produce nanostructures having desired properties. For example, feed solutions having higher concentrations of structural materials can produce thicker structures, such as nanotubes having thicker sheaths. As a particular example, a feed rate of 0.03 mL/hour with a 1:10 weight ratio of metal to solvent can produce fibers having an average inner diameter of about 200 nm and a wall thickness of about 50 nm. The feed rates can also be used to vary the properties of the nanostructures. For example, higher flow rates of the feed solution can be used to produce structures having larger diameter lumens. In some examples, the feed rate of the structural solution is between about 0.1 ml/h and about 5 ml/h, such as between about 0.5 ml/h and about 1 ml/h or about 0.6 ml/h. Examples of feed rates for the core material are between about 0.01 ml/h and about 2 ml/h, such as about 0.05 ml/h to about 0.3 ml/h.

Suitable potentials, in some examples, are between about 2 kV and about 100 kV, such as between about 5 kV and about 30 kV or about 5 kV to about 20 kV. A suitable high-voltage power supply is the ES30P-5W, available from Gamma High Voltage Research, Inc., of Ormond Beach, Fla. The electrodes, in some examples, are placed between about 2 cm to about 30 cm from the outlets of the feed solutions, such as between about 5 cm and about 20 cm. The spinning process is typically carried out a temperature of between about 0° C. and about 50° C., such as between about 15° C. and 30° C. The temperature can be adjusted depending on the volatility and viscosity of solutions used in the spinning process.

Once the nanostructures are formed, the sheath material can be removed, such as by calcining the structures, such as at about 250° C. to about 800° C., such as about 500° C., for about 15 minutes to about 5 hours, such as for about one hour. The sheath material can also be extracted using a suitable solvent. Suitable solvents include non-polar organic solvents, such as alkanes, aromatic solvents, petroleum ether, or mixtures thereof. In a particular example, the core material is extracted using octane.

Functionalization of TiO₂ nanotubes 12 can be carried out by loading the so-formed nanotubes 12 with the desired electron mediator 14, such as palladium, by means and methods known in the art. In one example, dried TiO₂ nanotube samples can be immersed in a palladium salt solution, e.g., 0.5 wt % PdCl₂ containing ethanolic solution, for 30 minutes under ultrasonication. The Pd containing solution wets the internal and external surfaces of the TiO₂ nanotubes almost thoroughly because of pre-drying and ultrasonication. The Pd salt loaded nanotube then may be vacuum dried to remove ethanol. After which, the samples can be annealed, for example, at 500° C. for 2 h in a reducing atmosphere containing 10% hydrogen in argon.

Non-limiting example of the systems 10, 100, and uses thereof, in accordance with the description are now disclosed below. These examples are merely for the purpose of illustration and are not to be regarded as limiting the scope of the invention or the manner in which it can be practiced. Other examples will be appreciated by a person having ordinary skill in the art.

### EXAMPLES

Photoelectrochemical and photocatalytic CO₂ reduction has been demonstrated using TiO₂ nanotubular arrays with and without Pd nanoparticles, which were illuminated with simulated solar light and without any external electrical energy.

Photoelectrochemical examples were carried out using band gap ˜2.2 eV modified TiO₂ nanotubular array as photo anode, pure Ti as cathode, and Ag/AgCl as reference electrode. The electrolyte was aqueous H₂SO₄ (pH: 4.5) saturated with CO₂ (the solution was continuously purged with CO₂ at a rate of ˜10 cc/min). The cathode was placed in a separate compartment and was connected to the anode compartment through a porous frit (Ace Glass type E pores). A computer controlled potentiostat (Model: SI 1286, Schlumberger, Farnborough, England) was employed to control the potential and record the photocurrent generated. Commercial grade CO₂ was continuously purged in the solution at least one hour before the start of the experiments to ensure CO₂ saturation. The potential of the TiO₂ sample was scanned from the open circuit potential to 0.5 V. The TiO₂ nanotubular samples were illuminated with a 300 W solar simulator (Model: 69911, Newport-Oriel Instruments, Stratford, Conn., USA). The intensity of the light was measured by a thermopile sensor (Model 70268, Newport) using a radiant power and energy meter (Model 70260, Newport Corporation, Stratford, Conn., USA).

For photocatalysts examples, the TiO₂ nanotube sample loaded with Pd nanoparticles was illuminated with simulated solar light and without any external electrical energy. Other details were similar to conditions used for photoelectrochemical conversion.

The nanotubular TiO₂ arrays in the examples were formed by anodization of 0.2 mm thick Ti foils (size 70×50 mm²) in fluoride (0.14 M NaF) containing 0.5 M phosphoric acid solution. A two-electrode configuration was used for anodization. Two larger Ti (75×75 mm²) sheets were kept on either side of the Ti sheet to be anodized and served as cathodes. The anodization was carried out at 20 V for about 1 h. After anodization, the samples were cleaned ultrasonically for about 45 seconds in ethanol solution, followed by rinsing in distilled water and dried in an electric oven at 100° C. for about an hour. Functionalization of TiO₂ nanotubes was carried out at this stage by loading with Pd. The dried TiO₂ samples were immersed in 0.5 wt % PdCl₂ containing ethanolic solution for 30 minutes under ultrasonication. The Pd containing solution wetted the internal and external surfaces of the TiO₂ nanotubes almost thoroughly because of pre-drying and ultrasonication. The Pd salt loaded samples were vacuum dried for about 12 h to remove ethanol. Then the samples were annealed at 500° C. for 2 h in a reducing atmosphere containing 10% hydrogen in argon. This heat treatment resulted in reduction of Pd(II) to Pd(O) nanoparticles loaded on to TiO₂ nanotubes. Furthermore, the amorphous TiO₂ nanotubes were transformed to crystalline (a mixture of anatase+rutile phases) nanotubes during the heat treatment at 500° C. This photo catalyst is hereafter referred to as PdTiO₂ NT in Table 1.

The nanotubes prepared by anodization in acidified fluoride solution have an inside diameter in the range of 80-100 nm and a length of about 400-600 nm. The wall thickness of the nanotubes was in the range of 15-25 nm. The Pd nanoparticies were found to be present uniformly on the walls of the nanotubes with a size distribution ranging from 3-14 nm. The weight percent of the Pd nanoparticles loaded onto the TiO₂ nanotubes was determined by TEM-EDX analyses of several nanotubes. The loading varied in the range of 0.8-1.2 wt % between the nanotubes.

In the examples using TiO₂ nanotubular arrays without Pd nanoparticles, the titania nanotube was further carbon modified to provide a carbon modified TiO₂₋ₓCₓ type nanotube, which is hereafter referred to as TiO₂₋ₓCₓ NT in Table 1 below. The TiO₂₋ₓCₓ NT nanotubes were prepared by sonoelectrochemcial anodization process of titanium metal using ammonium fluoride, sodium salt of EDTA, ethylene glycol and water. Crystallization of the nanotubes is carried out at 500° C. under nitrogen atmosphere. Nanotubes prepared by this method may be in the range of 500 nm to 100 μm long with pore diameters 10-250 nm.

The electrolyte, or electrically conductive fluid, was analyzed at regular intervals during the catalytic processes using UV-VIS photospectrometry to measure reduction products such as CH₃OH, HCHO, HCOOH, etc. The pre-calibrated optical absorption data of known volume fractions of methanol in the solution were used for calculating the rate of methanol conversion in this investigation. Table 1 shows the yield of methanol using various catalysts. It was observed that both photoelectrochemical and photocatalytic reduction processes predominantly formed methanol (CH₃OH). A TiO₂ nanotubular photoanode used with a p-type compound semiconductor photo cathode may have higher selectivity for CH₃OH and greater energy conversion efficiency.

The conversion rates are noted in Table 1 below. The system may be arranged in parallel fashion in an effort to increase the conversion, such as up to 100%. The conversion rates may also be increased by increasing the size of the catalysts (4 cm² electrodes were used to obtain the disclosed results).

TABLE 1
Methanol production from CO₂^{a}

| | | Methanol | Conversion |
|---|---|---|---|
| Catalysts | Conditions | yield^{b} | rate |
| No | Solar light | No product | 0% |
| catalyst | | | |
| TiO₂₋ₓCₓ | Photoelectrochemical, solar | 0.016 mol/hr | 61.5% |
| NT | light | | |
| PdTiO₂ | Photo catalytic, solar light | 0.012 mol/hr | 50.0% |
| NT | | | |

^{a}Examples carried out in aqueous H₂SO₄ solution (pH 4.5) in a continuous flow reactor
^{b}Yield was calculated from UV spectroscopy

Table 1 summarizes the results obtained using functionalized TiO₂ nanotubes for the reduction of CO₂ to methanol. The reaction did not occur without any catalyst. In the presence of doped TiO₂ nanotubes, i.e., TiO₂₋ₓCₓ NT, under photoelectrochemical conditions, the reaction produced 0.017 mol/hr methanol. Under photocatalytic conditions, the PdTiO₂ NT produced 0.012 mol/hr methanol. These results indicate that these types of catalysts can be used under both photoelectrochemical and photocatalytic conditions for the conversion of carbon dioxide to liquid organic fuels.

While the present invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicant's general inventive concept.

What is claimed is:

1. A system for converting carbon dioxide to an organic compound, the system comprising:
an array of nanotubes including nanoparticles of an electron mediator dispersed on a surface of the nanotubes;
an electrically conductive fluid, the array of nanotubes being at least partially immersed in the electrically conductive fluid;
a light source that irradiates the array of nanotubes;
a source of carbon dioxide; and
an inlet for delivering the carbon dioxide to the electrically conductive fluid whereat at least a portion of the carbon dioxide is converted to a different organic compound via contact with an irradiated array of nanotubes.

2. The system of claim 1 wherein the nanotubes are titania nanotubes.

3. The system of claim 2 wherein the titania nanotubes have a band gap of between about 2.0 ev and about 2.2 ev.

4. The system of claim 2 wherein the electron mediator is palladium.

5. The system of claim 4 wherein the electrically conductive fluid is a dilute sulfuric acid solution or an imidazolium salt solution.

6. The system of claim 1 wherein the light source irradiates visible light.

7. The system of claim 1 wherein the carbon dioxide is converted to methanol via contact with the irradiated array of nanotubes.

8. The system of claim 1 further comprising a distillation unit, wherein the distillation unit distills the organic compound from the electrically conductive fluid.

9. The system of claim 8 further comprising a dehydration catalyst that converts at least a portion of the distilled organic compound to another organic compound.

10. A system for converting carbon dioxide to an organic compound, the system comprising:
an anode including an array of nanotubes;
a cathode including an electrically conductive material, the cathode cooperates with the anode to receive electrons from the anode;
an electrically conductive fluid, the anode and cathode being at least partially immersed in the electrically conductive fluid;
a light source that irradiates at least the anode;
a source of carbon dioxide; and
an inlet for delivering the carbon dioxide to the electrically conductive fluid whereat at least a portion of the carbon dioxide is converted to a different organic compound via contact with the anode, cathode, or both.

11. The system of claim 10 wherein the nanotubes are titania nanotubes.

12. The system of claim 11 wherein the titania nanotubes are carbon modified.

13. The system of claim 11 wherein the titania nanotubes have a band gap of between about 2.0 ev and about 2.2 ev.

14. The system of claim 10 wherein the nanotubes are titania nanotubes including nanoparticles of an electron mediator dispersed on a surface of the nanotubes.

15. The system of claim 14 wherein the electron mediator is palladium.

16. The system of claim 15 wherein the electrically conductive fluid is a dilute sulfuric acid solution or an imidazolium salt solution.

17. The system of claim 10 wherein the light source irradiates visible light.

18. The system of claim 10 wherein the carbon dioxide is converted to methanol.

19. The system of claim 10 wherein the electrically conductive material is a semiconductor material.

20. The system of claim 10 wherein the cathode defines a gas-diffusing p-type semiconductor including a titanium dioxide substrate.

21. The system of claim 10 further comprising a distillation unit, wherein the distillation unit distills the organic compound from the electrically conductive fluid.

22. The system of claim 21 further comprising a dehydration catalyst that converts at least a portion of the distilled organic compound to another organic compound.

23. The system of claim 10 wherein the system further includes a reference electrode to define a three electrode cell.

24. A method for converting carbon dioxide to an organic compound, the method comprising:
irradiating an array of nanotubes at least partially immersed in an electrically conductive fluid, the array of nanotubes including nanoparticles of an electron mediator that are dispersed on a surface of the nanotubes; and
delivering carbon dioxide to the electrically conductive fluid whereat at least a portion of the carbon dioxide is converted to a different organic compound via contact with the irradiated array of nanotubes.

25. The method of claim 24 further comprising distilling the organic compound from the electrically conductive fluid.

26. The method of claim 25 further comprising converting at least a portion of the organic compound to another organic compound.

27. The method of claim 26 wherein distilling the organic compound from the electrically conductive fluid comprises distilling methanol from the electrically conducting, and wherein converting at least a portion of the organic compound to another organic compound comprises converting at least a portion of the methanol to dimethyl ether via contact with a dehydration catalyst.

28. The method of claim 24 wherein the nanotubes are titania nanotubes.

29. The method of claim 28 wherein the titanic nanotubes have a band gap of between about 2.0 ev and about 2.2 ev.

30. The method of claim 24 wherein irradiating an array of nanotubes at least partially immersed in an electrically conductive fluid comprises irradiating with visible light the array of nanotubes at least partially immersed in the electrically conductive fluid.

31. A method for converting carbon dioxide to an organic compound, the method comprising:
irradiating an anode including an array of nanotubes, the anode being at least partially immersed in an electrically conductive fluid;
supplying electrons from the irradiated anode to a cathode including an electrically conductive material, the cathode being at least partially immersed in the electrically conductive fluid; and
delivering carbon dioxide to the electrically conductive fluid whereat at least a portion of the carbon dioxide is converted to a different organic compound via contact with the anode, cathode, or both.

32. The method of claim 31 further comprising distilling the organic compound from the electrically conductive fluid.

33. The method of claim 32 further comprising converting at least a portion of the organic compound to another organic compound.

34. The method of claim 33 wherein distilling the organic compound from the electrically conductive fluid comprises distilling methanol from the electrically conducting, and wherein converting at least a portion of the organic compound to another organic compound comprises converting at least a portion of the methanol to dimethyl ether via contact with a dehydration catalyst.

35. The method of claim 31 wherein the nanotubes are titania nanotubes.

36. The method of claim 31 wherein the titania nanotubes have a band gap of between about 2.0 ev and about 2.2 ev.

37. The method of claim 31 wherein the nanotubes are titania nanotubes including nanoparticles of an electron mediator dispersed on a surface of the nanotubes.

38. The method of claim 31 wherein irradiating an anode including an array of nanotubes comprises irradiating with visible light the anode including an array of nanotubes.

39. The method of claim 31 wherein the electrically conductive material is a semiconductor material.

40. The method of claim 31 wherein the cathode defines a gas-diffusing p-type semiconductor including a titanium dioxide substrate.
